(19) United States
(12) Patent Application Publication
(10) Pub. No.: US 2008/0177312 A1
(43) Pub. Date: Jul. 24, 2008

(54) Interspinous Process Spacer Device

(75) Inventors: Miguelangelo J. Perez-Cruet, Bloomfield, MI (US); John R. Pepper, Cheshire, CT (US); John A. Miller, Bloomfield Village, MI (US)

Correspondence Address: MILLER IP GROUP, PLC;MI4 SPINE, LLC, 42690 WOODWARD AVE., SUITE 200, BLOOMFIELD HILLS, MI, 48304, US

(73) Assignee: MI4SPINE, LLC, Bloomfield Village, MI (US)

(21) Appl. No.: 11/738,211

(22) Filed: Apr. 20, 2007

Related U.S. Application Data
(63) Continuation-in-part of application No. 11/646,749, filed on Dec. 28, 2006.

(51) Int. Cl. A61B 17/70

(52) U.S. Cl. 606/249

(57) ABSTRACT

A spacer device that is to be inserted between the spinous process of adjacent vertebrae. In one embodiment, the spacer device is percutaneously inserted between the spinous process of adjacent vertebrae using minimally invasive surgical procedures. The spacer device includes a body portion having a channel extending therethrough, a plate member attached at one end of the body portion that is larger cross-wise than the body portion, and at least one retaining member attached proximate to an opposite end of the body portion from the plate member. The spacer device is inserted between the spinous process with the retaining member stored within the body portion. A deploying device is inserted into the channel to deploy the retaining member to lock the spacer device in place.

### CROSS-REFERENCE TO RELATED APPLICATIONS

This application is a continuation-in-part application of U.S. patent application Ser. No. 11/646,749, filed Dec. 28, 2006, titled “Minimally Invasive Interspinous Process Spacer Insertion Device.”

### BACKGROUND OF THE INVENTION

1. Field of the Invention

This invention relates generally to a spacer device inserted between the spinous process of adjacent vertebrae and, more particularly, to an interspinous process spacer device that is percutaneously inserted between the spinous process of adjacent vertebrae using minimally invasive surgical procedures.

2. Discussion of the Related Art

The human spine includes a series of vertebrae interconnected by connective tissue referred to as discs that act as a cushion between the vertebrae. The discs allow for movement of the vertebrae so that the back can bend and rotate. The vertebra includes a bony spinous process that protrudes towards the back.

The intervertebral disc is an active organ in which the normal and pathologic anatomies are well known, but the normal and pathologic physiologies have not been greatly understood. The intervertebral disc permits rhythmic motions required of all vertebrate animals in their various forms of locomotion. The disc is a high-pressure system composed primarily of absorbed water, an outer multilayered circumferential annulus of strong, flexible, but essentially inelastic collagen fibers, and an inner core of a hydrogel called the nucleus pulposus. The swelling of the contained hydrogel creates the high pressure that tightens the annular fibers and its laminations. Degeneration of discs in humans is typically a slow, complex process involving essentially all of the mechanical and physiologic components with loss of water holding capacity of the disc. Discogenic pain arises from either component, but is primarily due to altered chemistry. When this pain is severely disabling and unyielding, the preferred contemporary treatments are primarily surgical, particularly fusion and/or disc replacement.

Annular collagen fibers are arranged in circumferential belts or laminations inserting strongly and tangentially in right- and left-handed angulated patches into each adjacent vertebral body. Inside the annular ring is contained an aggrecan, glycosaminoglycan, a protein-sugar complex gel having great hygroscopic ability to hold water. The swelling pressure of this gel of the nucleus maintains the pressure within the annulus, forcing the vertebrae apart and tightening the annular fibers. This tightening provides the primary mechanical stability and flexibility of each disc of the spinal column. Further, the angulated arrangement of the fibers also controls the segmental stability and flexibility of the motion segment. Therefore, the motion of each segment relates directly to the swelling capacity of the gel and secondarily to the tightness of intact annulus fibers. The same gel is also found in thin layers separating the annular laminar construction, providing some apparent elasticity and separating the laminations, reducing interlaminar torsional abrasion. With aging or degeneration, nucleus gel declines, while collagen content, including fibrosis, increases.

Disc degeneration, which involves matrix, collagen and aggrecan, usually begins with annular tears or alterations in the endplate nutritional pathways by mechanical or pathophysiologic means. However, the disc ultimately fails for cellular reasons. As a person ages, the discs in the spine go through a degenerative process that involves the gradual loss of the water holding capacity of the disc, referred to as desiccation. As a result of this loss of water, the disc space height may partially collapse, which may lead to chronic back pain disorders and/or leg pain as a result of the nerves being pinched.

Progressive injury and aging of the disc occurs normally in later life and abnormally after trauma or metabolic changes. In addition to the chemical effects on the free nerve endings as a source of discogenic pain, other degenerative factors may occur. Free nerve endings in the annular fibers may be stimulated by stretching as the disc degenerates, bulges, and circumferential delamination of annular fibers occurs. This condition may lead to a number of problems, such as back pain. It has been shown that a person's disc is typically taller in the morning when a person awakes. This phenomenon may be due in part to the reduction of body weight forces on the disc when lying in a recumbent position overnight that causes the disc height to restore. Therefore, reduction of compressive forces on the disc may help to restore disc space height.

As discussed above, as a person ages, the discs of the spine degenerate, and the disc space height collapses. Further, the ligaments and facets of the spine degenerate as well. These problems lead to a reduction in the foramenal height of the vertebrae, often causing central or lateral canal stenosis. The foramen is the opening between the vertebrae that allows the nerve from the spinal cord to pass through. Because the nerve passes through the foramen, the nerve will often get pinched leading to various types of back pain. Further, these problems often lead to difficulty to walking. Additionally, the lateral canal stenosis causes the nerve to get pinched in the spinal canal. These conditions often lead to neurogenic claudication, where the patient typically responds by walking shorter distances, then sitting down, and then flexing the spine by leaning over or by walking with the aid of a device, which helps to flex the spine.

Current surgical procedures that exist for addressing this pathology require that the ligaments and bone that are causing the compression be removed surgically to take the pressure off of the nerves. Recently, interspinous process spacers, such as the X-stop, have been developed. Known interspinous process spacers operate by flexing the spine and opening the canal, lateral recess and foramen to take pressure off of the nerves. These devices typically can be useful for conditions of lateral recess stenosis or foramenal stenosis alone. These devices can also be potentially useful as an adjunct to minimally invasive laminectomy for stenosis where the spinous process is preserved. Interspinous process spacers can act as an adjunct device to minimally invasive laminectomy for stenosis to treat the foramenal stenosis component of this disorder. Following minimally invasive lumbar laminectomy for stenosis, the interspinous process spacer could be placed between the preserved spinous processes of the spine. The result would be to address and treat the lateral or foramenal stenosis that could persist despite the decompression of the spinal canal.

### SUMMARY OF THE INVENTION

In accordance with the teachings of the present invention, a spacer device is disclosed that is inserted between the spinous process of adjacent vertebrae. In one embodiment, the spacer device is percutaneously inserted between the spinous process of adjacent vertebrae using minimally invasive surgical procedures. In one non-limiting embodiment, the spacer device includes a body portion having a channel extending therethrough, a plate member attached at one end of the body portion that is larger cross-wise than the body portion, and at least one retaining member attached proximate to an opposite end of the body portion from the plate member. The spacer device is inserted between the spinous process with the retaining member stored within the body portion. A deploying device is inserted into the channel to deploy the retaining member to lock the spacer device in place.

Additional features of the present invention will become apparent from the following description and appended claims, taken in conjunction with the accompanying drawings.

### BRIEF DESCRIPTION OF THE DRAWINGS

FIG. 1 is a side view of a minimally invasive interspinous process spacer insertion device in a retracted position, according to an embodiment of the present invention;

FIG. 2 is a perspective view of an interspinous process spacer, according to an embodiment of the present invention;

FIG. 3 is a side view of the device depicted in FIG. 1 showing an advanced arced trocar rod;

FIG. 4 is a side view of the device depicted in FIG. 1 showing an advanced curved cannulated sleeve;

FIG. 5 is a side view of the device depicted in FIG. 1 with the arced trocar rod removed and the cannulated sleeve positioning the spacer between two spinous process;

FIG. 6 is a side view of the device depicted in FIG. 1 including a flexible driver positioned within the cannulated sleeve for rotating the interspinous process spacer;

FIG. 7 is a side view of the interspinous process spacer positioned between the spinous process of adjacent vertebrae;

FIG. 8 is a broken-away view of the end of the cannulated sleeve;

FIG. 9 is another perspective view of the interspinous process spacer;

FIG. 10 is a side view of the flexible driver shown in FIG. 6;

FIG. 11 is a perspective view of a minimally invasive interspinous process spacer insertion device including two advanced arced trocar rods, according to another embodiment of the present invention;

FIG. 12 is a perspective view of an interspinous process spacer, according to another embodiment of the present invention;

FIG. 13 is a perspective view and FIG. 14 is a side view of an interspinous process spacer, according to another embodiment of the present invention;

FIGS. 15 and 16 are cross-sectional views of an interspinous process spacer showing retaining members in a stored position and a deployed position, respectively, according to an embodiment of the present invention;

FIGS. 17 and 18 are cross-sectional views of an interspinous process spacer showing retaining members in a stored position and a deployed position, respectively, according to another embodiment of the present invention;

FIGS. 19 and 20 are perspective views of an interspinous process spacer device showing the device in a lowered position and a raised position, respectively, according to another embodiment of the present invention;

FIGS. 21, 22 and 23 are perspective views of an interspinous process spacer showing a retaining member in a retracted position, a partially deployed position and a fully deployed position, according to another embodiment of the present invention; and

FIGS. 24 and 25 are perspective views of an interspinous process spacer showing retaining members in a retracted position and in a deployed position, respectively, according to another embodiment of the present invention.

### DETAILED DESCRIPTION OF THE EMBODIMENTS

The following discussion of the embodiments of the invention directed to an interspinous process spacer device to be positioned between the spinous process of adjacent vertebra is merely exemplary in nature, and is in no way intended to limit the invention or its applications or uses.

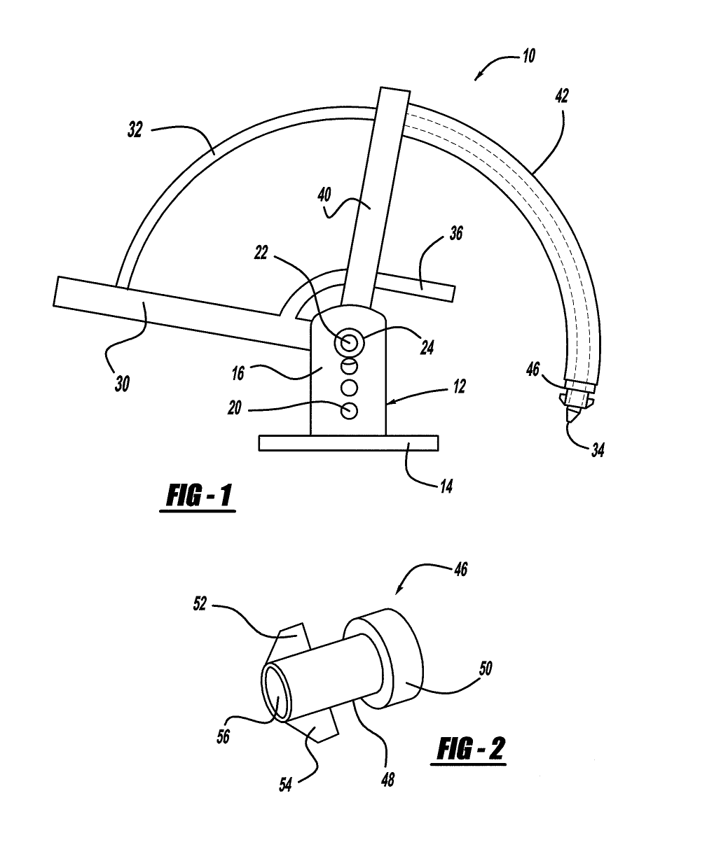

FIG. 1 is a side view of a minimally invasive interspinous process spacer insertion device 10, according to an embodiment of the present invention. As will be discussed in detail below, the device 10 is used to insert an interspinous process spacer between the interspinous process of adjacent vertebrae in a minimally invasive surgical procedure to provide a minimally invasive surgical solution to foramenal stenosis and/or lateral or central canal stenosis by opening up the foramen and spinal canal by flexing the spine and indirectly decompressing the neural elements. The device 10 preserves the muscle attachments to the spine, as well as integrity of the interspinous process ligament, and can be potentially performed under local anesthesia.

The device 10 includes a base portion 12 having a base plate 14 and a pair of opposing spaced apart plate stanchions 16. The stanchions 16 include a series of holes 20 that provide a height adjustment for the device 10, as will become apparent from the discussion below. A support rod 22 including a knob 24 is inserted within opposing holes 20 in the stanchions 16. The support rod 22 also extends through a hole in a cylindrical trocar arm 30 so that the trocar arm 30 is rotatably movable relative to the base portion 12. The trocar arm 30 includes an arced trocar rod 32 having a trocar tip 34. The trocar rod 32 is removably mounted to the trocar arm 30 in any suitable manner, such as by threads, snap fit, etc. The trocar arm 30 also includes a hard stop arm 36 that will contact the base plate 14 to prevent the trocar rod 32 from advancing beyond a maximum position.

The device 10 also includes a cylindrical cannulated arm 40 having an opening (not shown) through which the support rod extends so that the arm 40 is also rotatably mounted to the base portion 12. The cannulated arm 40 also includes another opening (not shown) through which the hard stop arm 36 can move. An arced cannulated sleeve 42 is rigidly coupled to the arm 40, and has the same curvature as the trocar rod 32. The cannulated sleeve 42 has a central bore through which the arced trocar rod 32 is positioned. An interspinous process spacer 46 is rotatably mounted to an end of the cannulated sleeve 42.

FIG. 2 is a perspective view of the spacer 46 removed from the cannulated sleeve 42. The spacer 46 includes a cylindrical body portion 48 and an end plate 50, where the body portion 48 has an internal bore 56. A pair of locking fins 52 and 54 are mounted to the body portion 48 opposite to the end plate 50, as shown. The spacer 46 can be made of any suitable material, such as a rigid plastic that is a single molded piece. In one embodiment, the spacer is radio-opaque so that it is visible on an X-ray so that the surgeon can determine if the spacer 46 is in the proper location.

FIG. 3 is a side view of the device 10 showing a first step of the surgical procedure. The base portion 12 is aligned with a patient's spine by any suitable process, where the base plate 14 rests on the patient's back. During the alignment process, using, for example, fluoroscopy and a K-wire, well known to those skilled in the art, the height of the device 10 is adjusted by selecting the proper hole 20 for the arms 30 and 40. The proper hole 20 is determined by passing a calibrated measuring K-wire to the proper depth at which the interspinous process spacer 46 will be placed. This is done to assure that the tip 34 of the trocar rod 32 does not pass too deep. An incision is made in the patient lateral to the spine and relative to the base portion 12. The trocar arm 30 is raised so that the sharp trocar tip 34 is inserted through the incision and transverses the soft tissue and muscle of the patient so that the tip 34 extends between the spinous process 60 of adjacent vertebrae 62, as shown.

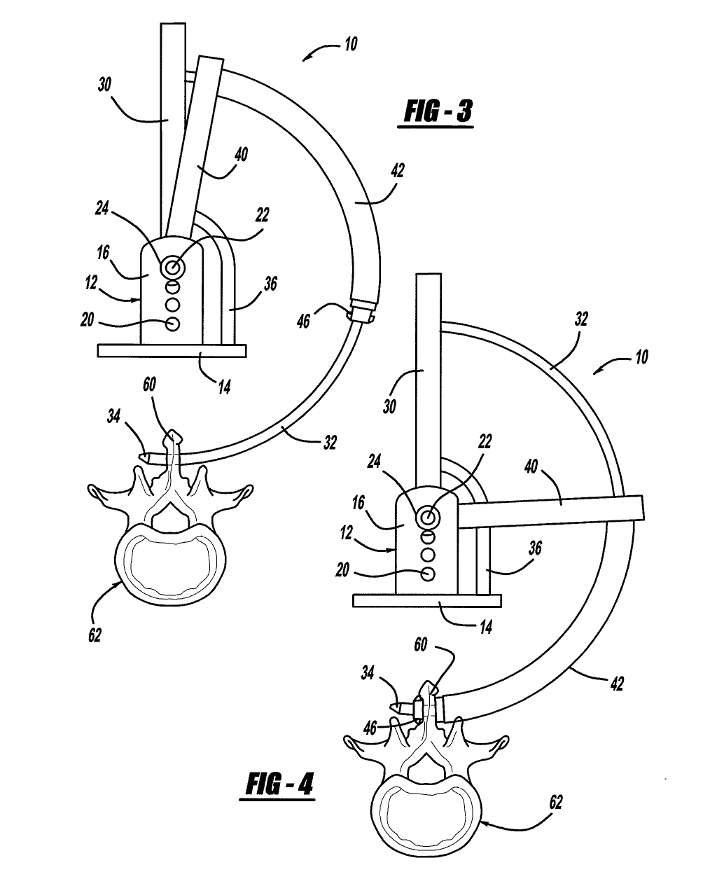

FIG. 4 shows a next step in the surgical procedure after the arm 32 has been extended between the spinous process 60. The surgeon will then rotate the arm 40 so that the cannulated sleeve 42 moves the spacer 46 down the trocar rod 32. The spacer 46 is coupled to the cannulated sleeve 42 so that the fins 52 and 54 are aligned in the proper orientation so that they easily slide between the spinous process 60. The spacer 46 is in the proper position, as shown in FIG. 4, when the fins 52 and 54 are on one side of the spinous process 60 and the end plate 50 is on the other side of the spinous process 60.

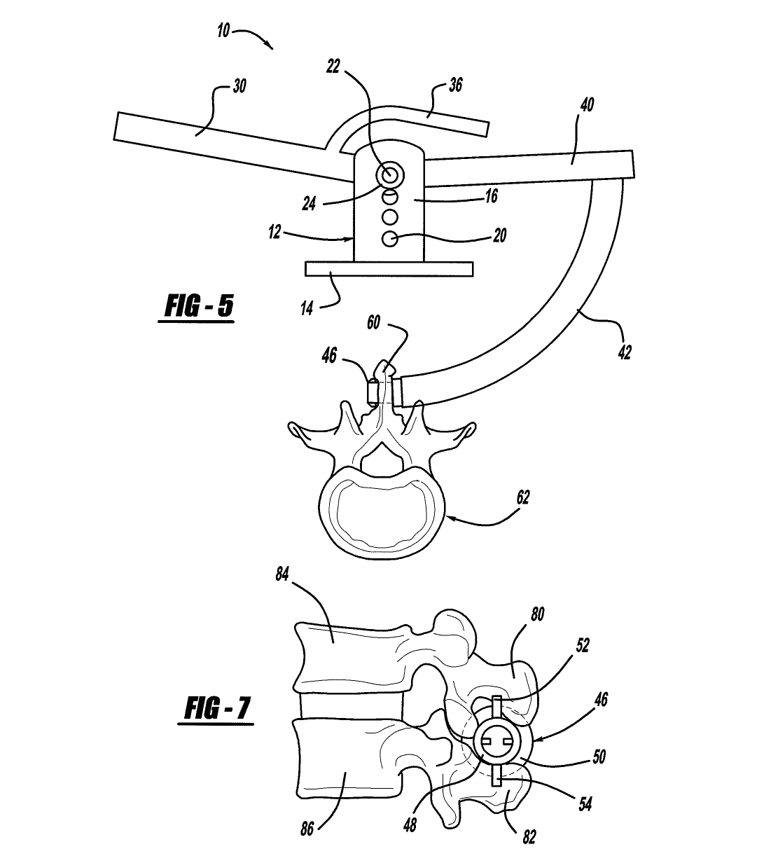

The arm 30 is then retracted so that the trocar rod 32 is removed from the patient. The arced trocar rod 32 is then removed from the trocar arm 30, as shown in FIG. 5.

FIG. 6 is a side view of the device 10 including a flexible driver 70 for rotating the spacer 46. The driver 70 includes a handle 72 and a flexible rod 74 mounted thereto. The rod 74 can be made of any suitable material, such as wound steel. The flexible rod 74 is inserted through the bore in the cannulated sleeve 42, and is coupled to the spacer 46. The rod 74 is rotated within the bore of the cannulated sleeve 42 to rotate the spacer 46 so that the fins 52 and 54 lock behind the spinous process 60 of the vertebra 62. The driver 70 is then detached from the spacer 46 and withdrawn from the cannulated sleeve 42. The arm 40 is then raised to remove the spacer 46 from the cannulated sleeve 42 and remove the cannulated sleeve 42 from the patient, where the spacer 46 remains in the patient between the spinous process 60.

In one embodiment, the spacer 46 can be placed after performing a minimally invasive spinous process preserving laminectomy for stenosis to aid in opening the foramen bilaterally.

In another embodiment, a drill head bit can be attached to the flexible rod 74 to drill off a portion of hypertrophied facet joints to allow for proper positioning of the spacer 46 at the base between the spinous processes. The drill head bit is then removed through the sleeve 42, and the spacer 46 is then placed.

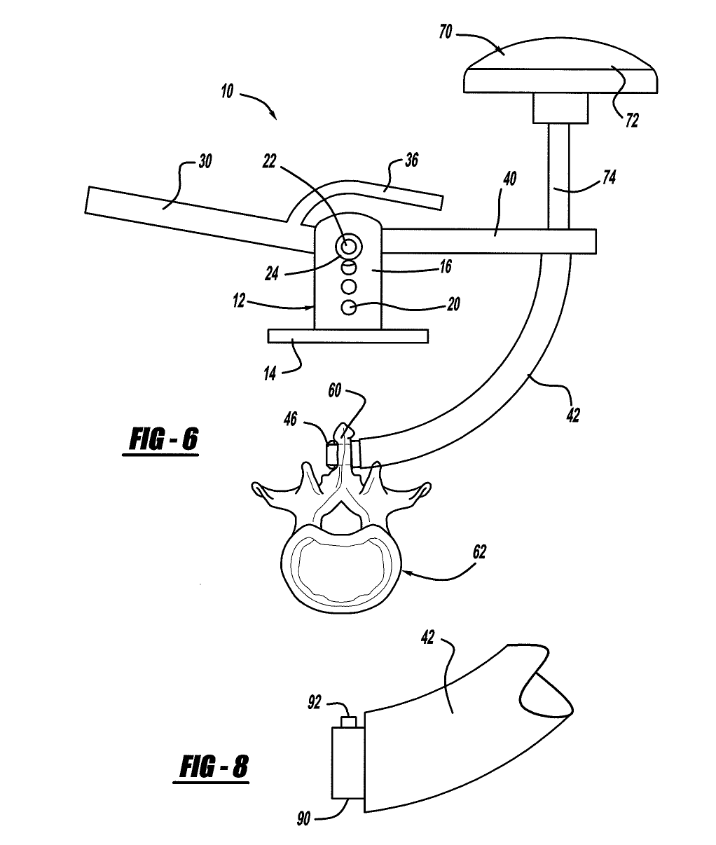

FIG. 7 is a side view of the spacer 46 positioned between the spinous process 80 and 82 of adjacent vertebrae 84 and 86, respectively, to separate the vertebrae 84 and 86 and provide relief for the stenosis. As is apparent, the fins 52 and 54 are positioned on one side of the spinous process 80 and 82 and the end plate 50 is positioned on an opposite side of the spinous process 80 and 82.

The spacer 46 can be attached to the cannulated sleeve 42 and the spacer 46 can be rotated once it is in position between the spinous process 60 in any effective or suitable manner for the purposes described herein. FIG. 8 is a broken-away side view of the cannulated sleeve 42 showing one non-limiting technique for attaching the spacer 46 thereto. The cannulated sleeve 42 includes a narrow-diameter end portion 90 that has an internal diameter that is about the same as the diameter of the internal bore 56 of the spacer 46. A hard stop pin 92 is attached to the narrow-diameter end portion 90.

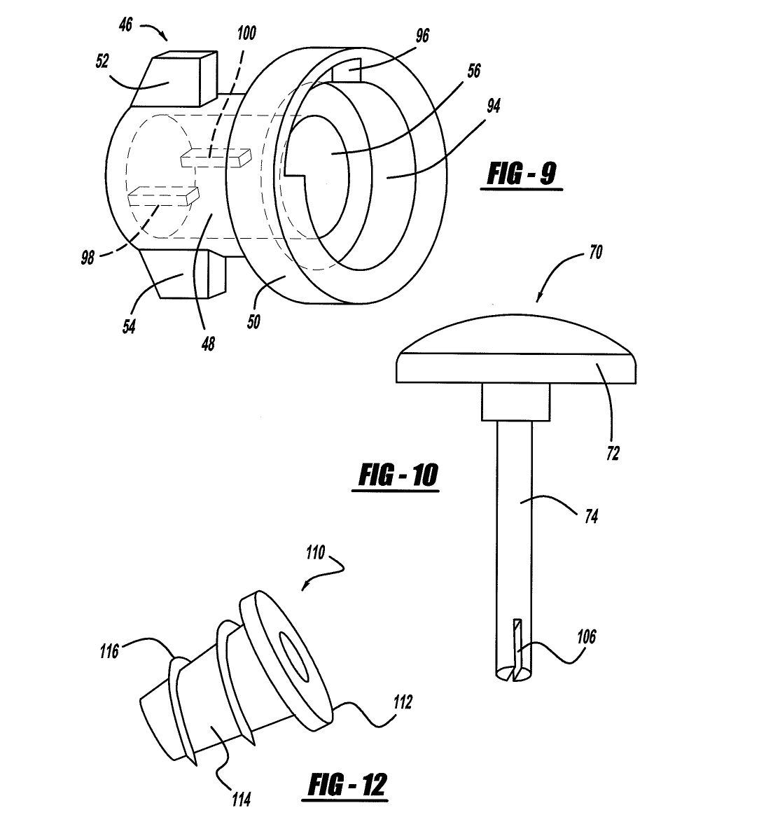

FIG. 9 is another perspective view of the spacer 46. A cylindrical bore 94 is provide within the end plate 50 that is concentric with the bore 56 and has a larger diameter than the bore 56. An arced slot 96 is provided within the end plate 50 adjacent to the bore 94, and covers about 90° of the circumference of the bore 94. The outer diameter of the end portion 90 is slightly less than the diameter of the bore 94 so that the spacer 46 can slide onto the end portion 90 and be held thereto in a friction type engagement. The end portion 90 can be slightly tapered to facilitate coupling of the spacer 46 to the cannulated sleeve 42. The hard stop pin 92 is positioned at the top end of the slot 96 so that the fins 52 and 54 are properly oriented relative to the insertion direction of the spacer 46 between the spinous process 60.

The spacer 46 includes a pair of opposing elongated tabs 98 and 100 extending partly across the internal bore 56, as shown. The height of the tabs 98 and 100 is such that they allow the arced trocar rod 32 to easily extend therebetween. The surgeon would be able to easily attach the spacer 46 to the narrow-diameter portion 90, and then slide the arced trocar rod 32 through the spacer 46 because this procedure would be performed outside of the patient.

FIG. 10 is a side view of the flexible driver 70 removed from the device 10. The driver 70 includes a slot 106 in the rod 74 at an opposite end to the handle 72. When the flexible driver 72 is extended down the cannulated arm 42, the surgeon aligns the slot 106 with the opposing tabs 98 and 100 so that the tabs 98 and 100 are positioned within the slot 106. The surgeon will then rotate the flexible driver 70, here in a clockwise direction, so that the spacer 46 rotates and the hard stop pin 92 moves along the arced slot 96. When the hard stop pin 92 hits the opposite end of the arced slot 96, the spacer 46 has been rotated 90°, and the fins 52 and 54 will be in the proper orientation for locking the spacer 46 between the spinous process 60. The flexible driver 70 can then be pulled off of the tabs 98 and 100 and be removed from the cannulated sleeve 42. Because the spacer 46 is now locked in placed, the surgeon can raise the arm 40 to detach the spacer 46 from the end portion 90 so that the spacer 46 remains in place.

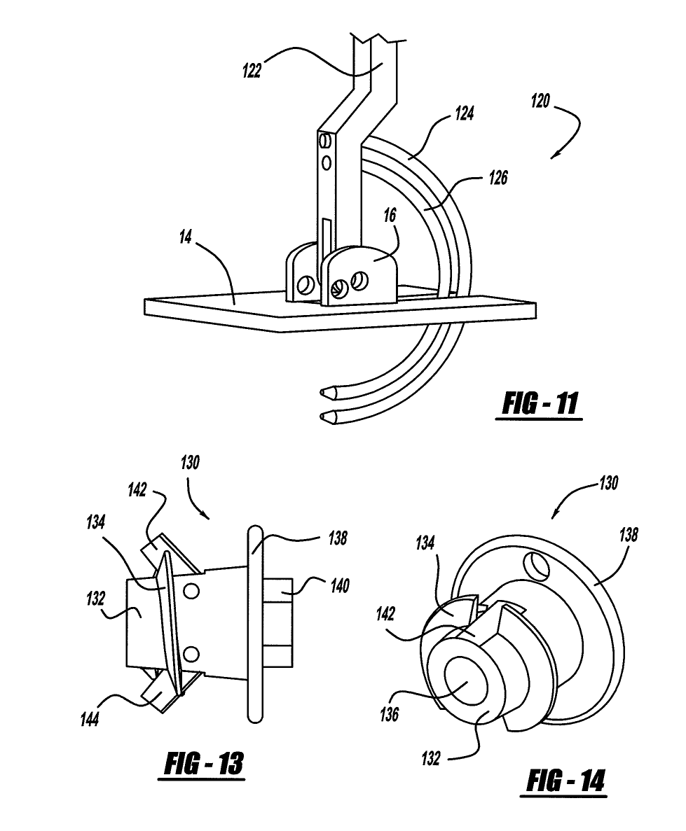

FIG. 11 is a perspective view of a minimally invasive interspinous process spacer insertion device 120 similar to the device 10 where like elements are identified by the same reference number, according to another embodiment of the present invention. The device 120 includes and trocar arm 122 and two advanced arced trocar rods 124 and 126 coupled thereto for placing two interspinous process spacers between the spinous process of adjacent vertebra.

Various spacer designs can be provided within the scope of the present invention. FIG. 12 is a perspective view of an alternate interspinous process spacer 110 including an end plate 112 and a tapered body portion 114. The body portion 114 includes threads 116 that allow the spacer 110 to be locked between the spinous process.

FIG. 13 is a perspective view and FIG. 14 is a side view of an interspinous process spacer 130, according to another embodiment of the present invention. The spacer 130 includes a tapered body portion 132 having a helical ridge 134 and a center bore 136. An annular rim 138 is attached to the body portion 132 and a coupling portion 140 is attached to the rim 138 opposite to the body portion 132. Opposing wing members 142 and 144 are pivotally attached to the body portion 132. The spacer 130 is placed by rotating the spacer 130 so that the ridge 134 pulls the spacer 130 between the spinous process until the rim 138 is positioned against one side of the spinous process. The wing members 142 and 144 are then extended to lock the spacer 130 to the spinous process.

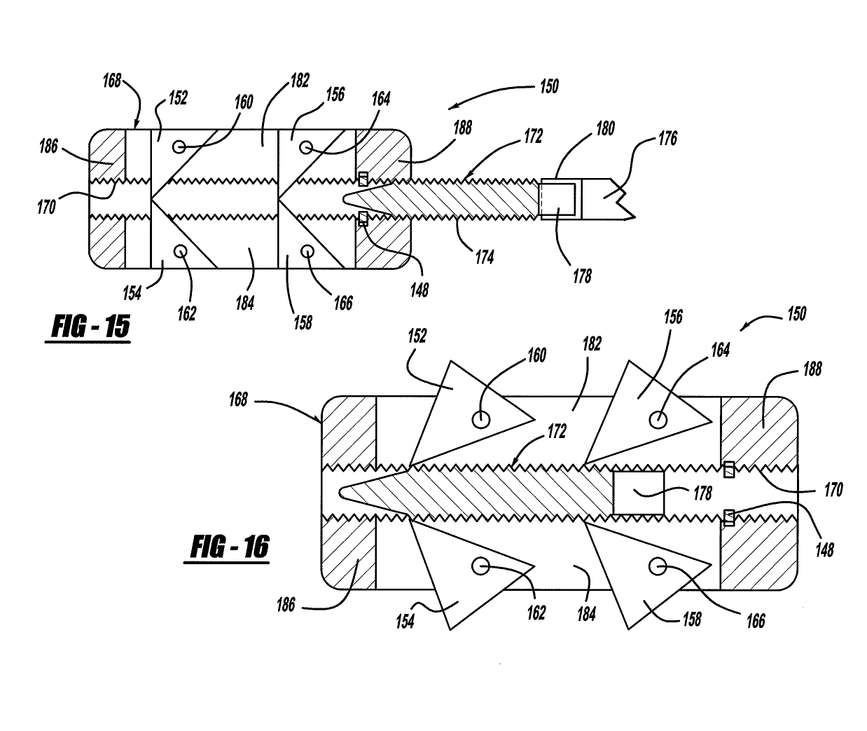

FIGS. 15 and 16 are cross-sectional views of an interspinous process spacer 150 including retaining members 152, 154, 156 and 158 shown in a stored position and a deployed position, respectively, according to another embodiment of the present invention. The retaining members 152, 154, 156 and 158 are triangular shaped members in this non-limiting design, where the member 152 is pivotally mounted to a body portion 168 of the spacer 150 by a pivot pin 160, the member 154 is pivotally mounted to the body portion 168 by a pin 162, the retaining member 156 is pivotally mounted to the body portion 168 by a pin 164 and the retaining member 158 is pivotally mounted to the body portion 168 by a pin 166. A threaded channel 170 extends through the body portion 168. A deploying device 172 including an outer threaded surface 174 is operable to be threaded through the channel 170. The deploying device 172 is releasably mounted to a flexible driver 176 of the type discussed above by inserting a cylindrical head 178 of the deploying device 172 into a cup 180 of the driver 176 in a friction engagement.

When the spacer 150 is inserted between the spinous process of adjacent vertebrae, such as in the manner as discussed above, the stored retaining members 152 and 154 will be at one side of the spinous process and the stored retaining members 156 and 158 will be at the other side of the spinous process. When the retaining members 152, 154, 156 and 158 are in the stored position, as shown in FIG. 15, the retaining members 152, 154, 156 and 158 extend across the channel 170. When the driver 176 is rotated, the deploying device 172 is threaded through the channel 170 and contacts the retaining members 152, 154, 156 and 158 causing them to pivot on the pivot pins 160, 162, 164 and 166, respectively. As the retaining members 152, 154 156 and 158 pivot, points of the retaining members 152, 154, 156 and 158 extend out of slots 182 and 184 between end portions 186 and 188 of the body portion 168. The deployed retaining members 152 and 154 will be at one side of the spinous process and the deployed retaining members 156 and 158 will be at the other side of the spinous process, thus, locking the spacer 150 in place. The driver 176 can be detached from the deploying device 172 by merely pulling on it, where the deploying device 172 stays within the channel 170 to hold the retaining members 152, 154, 156 and 158 in the deployed position. A flexible ring 148, or other member, can be used to prevent the deploying device 172 from backing out of the channel 170.

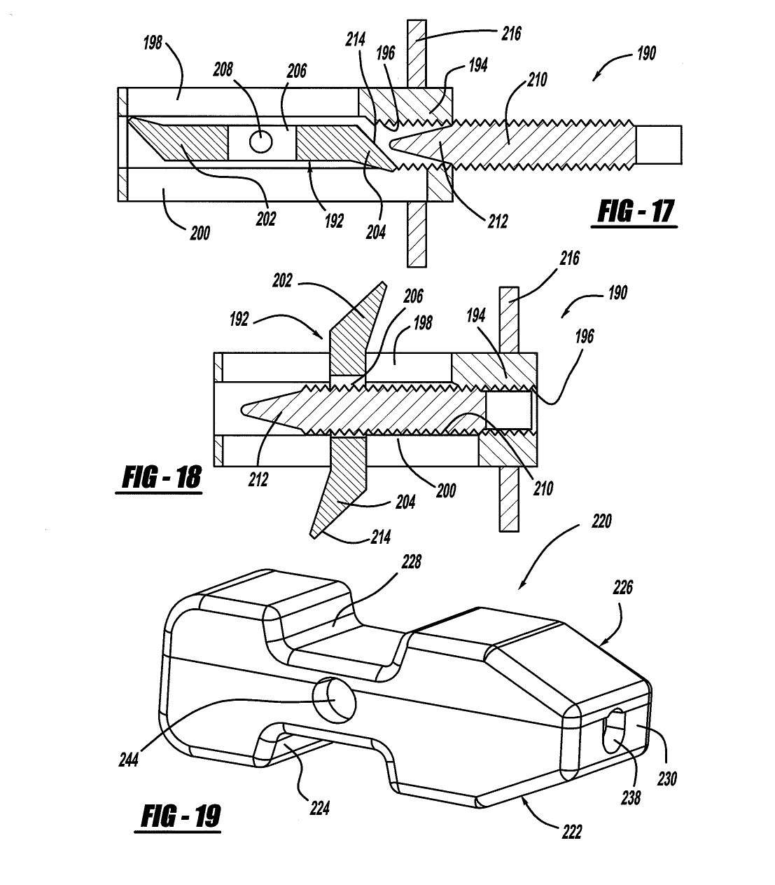

FIGS. 17 and 18 are cross-sectional views of an interspinous process spacer 190 showing a retaining member 192 in a stored position and a deployed position, respectively, according to another embodiment of the present invention. The spacer 190 includes a body portion 194 having a channel 196 extending therethrough. A pair of opposing slots 198 and 200 is provided in the body portion 194 so as to allow an angled first end 202 of the retaining member 192 to extend through the slot 198 and an angled second end 204 of the retaining member 192 to extend through the slot 200. The retaining member 192 includes a mounting plate 206 pivotally mounted to the body portion 194 by a pin 208 between the slots 198 and 200.

A deploying device 210 is threaded through the channel 196, and can be rotated by the flexible driver 176 as discussed above. When a tip 212 of the deploying device 210 contacts an angled edge 214 of the second end 204, the tip 212 rides along the angled edge 214, and causes the retaining member 192 to rotate on the pin 208 to the deployed position shown in FIG. 18 until the deploying device 210 is positioned across the plate 206 between the ends 202 and 204.

When the spacer 190 is positioned between the spinous process of adjacent vertebrae, such as by the procedure discussed above, an annular rim 216 is positioned at one side of the spinous process and the stored retaining member 192 is positioned at an opposite side of the spinous process. The deploying device 210 is then threaded through the channel 196 to cause the retaining member 192 to rotate to the deployed position where the position of the ends 202 and 204 are at an opposite side of the spinous process from the rim 216 to lock the spacer 190 in the proper position.

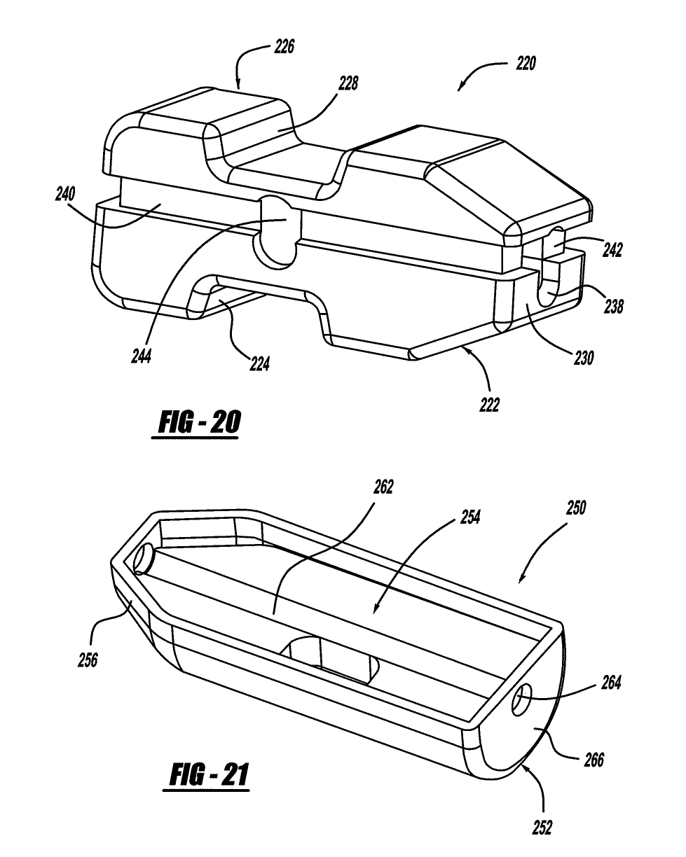

FIGS. 19 and 20 are perspective views of an interspinous process spacer 220 shown in a stored position and a deployed position, respectively, according to another embodiment of the present invention. The spacer 220 includes a lower body portion 222 having a central recess 224 and an upper body portion 226 having a central recess 228. The upper and the lower body portions 226 and 222 are angled to provide a narrow end portion 230. The interspinous process spacer 220 is inserted between the spinous process of adjacent vertebrae by directing the end portion 230 between the spinous process, for example, using the insertion device 10, so that the spinous process of one vertebrae is positioned proximate the recess 224 and the spinous process of the other vertebrae is positioned proximate the recess 228. When the spacer 220 is in the proper position, a suitable deploying device (not shown) is inserted into a channel 238 extending through and between the body portions 222 and 226. Rotation of the deploying device causes the upper body portion 226 to raise on rails 240 and 242 mounted within suitable slots in the lower body portion 222 so that the spinous process of the adjacent vertebra are positioned within the recesses 224 and 228 and the spacer 220 is locked in place. The spacer 220 can be provided with a transverse cannulated opening 244 that extends through and between the body portions 222 and 226, as shown.

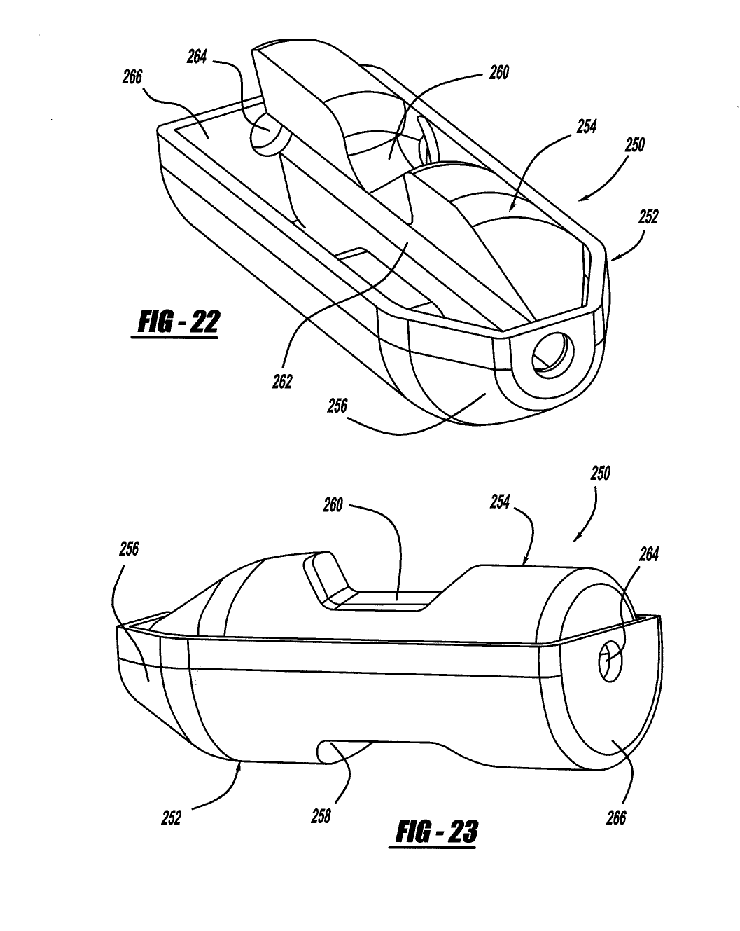

FIGS. 21, 22 and 23 are perspective views of an interspinous process spacer 250 shown in a stored position, an intermediate deployed position and a fully deployed position, respectively, according to another embodiment of the present invention. The spacer 250 includes an outer body portion 252 and an inner body portion 254 each having a general cylindrical shape. One end 256 of the outer body portion 252 is tapered so as to allow the spacer 250 to be easily inserted between the spinous process of adjacent vertebrae. The outer body portion 252 includes a recess 258 and the inner body portion 254 includes a recess 260 that operate in the same manner as the recesses 224 and 228. The inner body portion 254 is rotatably mounted to the outer body portion 252 and includes a channel 262 extending therethrough.

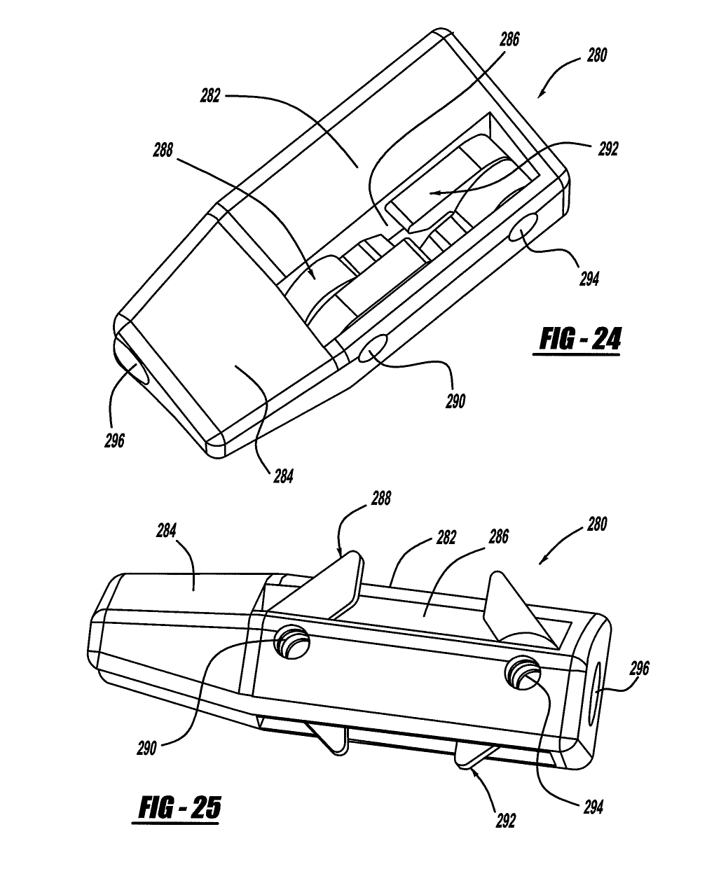

When the spacer 250 is positioned between the spinous process of adjacent vertebrae, such as by the process discussed above, the inner body portion 254 is completely enclosed within the outer body portion 252, as shown in FIG. 21. A suitable deploying device (not shown) is inserted through an opening 264 in an end plate 266 of the outer body portion 252 and into the channel 262. The device is rotated to cause the inner body portion 254 to rotate through the intermediate position as shown in FIG. 22 to the fully deployed position as shown in FIG. 23 to lock the spacer 250 in place.

FIGS. 24 and 25 are perspective views of an interspinous process spacer 280 showing the spacer 280 in a stored position and a deployed position, respectively, according to another embodiment of the present invention. The spacer 280 includes a body portion 282 having a tapered end portion 284 where a slot 286 is provided in and extends through the body portion 282. A first scissor device 288 is mounted to a pin 290 within and at one end of the slot 286, and a second scissor device 292 is mounted to a pin 294 within and at an opposite end of the slot 286. A channel 296 extends through the body portion 282 adjacent to the slot 286. The spacer 280 is positioned between the spinous process of adjacent vertebrae by inserting the tapered end 284 between the spinous process, such as by the process discussed above. The scissor devices 288 and 292 are closed and enclosed within the slot 286 when the spacer 280 is being inserted. When the spacer 280 is in the proper position, a suitable deploying device (not shown) is inserted down the channel 296 and is rotated to cause the scissor devices 288 and 292 on the pins 290 and 294 to open to the deployed position shown in FIG. 25, respectively.

The foregoing discussion discloses and describes merely exemplary embodiments of the present invention. One skilled in the art will readily recognize from such discussion and from the accompanying drawings and claims that various changes, modifications and variations can be made therein without departing from the spirit and scope of the invention as defined in the following claims.

What is claimed is:

1. An interspinous process spacer device comprising:
a body portion including a channel extending therethrough;
a plate member attached to the body portion, said plate member being larger cross-wise than the body portion; and
at least one retaining member mounted to the body portion, said at least one retaining member being movable from a stored position where the at least one retaining member is substantially enclosed within the body portion to a deployed position where the at least one retaining member extends from the body portion to provide a space between the at least one retaining member and the plate member in which an spinous process can be positioned.

2. The device according to claim 1 wherein the body portion is a cylindrical body portion and the plate member is an annular plate member.

3. The device according to claim 1 wherein the body portion is tapered where a larger diameter part of the body portion is at an end of the body portion including the plate member.

4. The device according to claim 1 wherein the at least one retaining member is two retaining members positioned on opposite sides of the body portion.

5. The device according to claim 4 wherein the retaining members are tapered so that a narrow end of the retaining members is closest to the end of the body portion opposite to the plate member and a wide end of the retaining members is closest to the plate member.

6. The device according to claim 4 wherein the retaining members are pivotally mounted to the body portion and are movable from a position that is flush with the body portion to a position that is extended from the body portion.

7. The device according to claim 1 wherein the at least one retaining member is a single retaining member having a center plate rotatably mounted to the body portion and end members extending therefrom, wherein the end members extend through opposing slots in the body portion when the retaining member moves from the stored position to the deployed position.

8. The device according to claim 7 wherein one of the end members includes an angled edge that receives a deploying apparatus that rides on the angled edge to cause the retaining member to rotate from the stored positioned to the deployed position.

9. The device according to claim 8 wherein the device further includes a threaded channel, said deploying apparatus being threaded through the threaded channel.

10. The device according to claim 1 wherein the spacer device is radio-opaque.

11. An interspinous process spacer device comprising:
a body portion including a channel extending therethrough; and
at least one retaining member pivotally attached to the body portion and extending into the channel, wherein the at least one retaining member pivots from a stored position substantially enclosed within the body portion to a deployed position where a portion of the at least one retaining member extends out of the body portion.

12. The device according to claim 11 wherein the at least one retaining member is a single retaining member having a center plate mounted to the body portion and end members extending therefrom, wherein the end members extend through opposing slots in the body portion when the retaining member moves from the stored position to the deployed position.

13. The device according to claim 12 wherein one of the end members includes an angled edge that receives a deploying apparatus that rides on the angled edge to cause the retaining member to rotate from the stored positioned to the deployed position.

14. The device according to claim 11 wherein the at least one retaining member is four retaining members where two of the retaining members are pivotally mounted to the body portion directly across the channel from each other and the other two retaining members are pivotally attached to the body portion directly across a channel from each other.

15. The device according to claim 14 wherein the retaining members are triangular in shape.

16. The device according to claim 11 wherein the channel is a threaded channel, and wherein a deploying apparatus is threaded through the threaded channel so as to deploy the at least one retaining member.

17. An interspinous process spacer device comprising:
a body portion including a threaded channel extending therethrough; and
a plurality of retaining members pivotally attached to the body portion and extending into the channel, wherein the plurality of retaining members pivot from a stored position substantially enclosed within the body portion to a deployed position through slots in the body portion where a portion of the plurality of retaining members extends out of the body portion, and wherein a deploying apparatus is threaded through the threaded channel so as to pivot and deploy the plurality of retaining members.

18. The device according to claim 17 wherein the plurality of retaining members is four retaining members where two of the retaining members are pivotally mounted to the body portion directly across the channel from each other and the other two retaining members are pivotally attached to the body portion directly across a channel from each other.

19. The device according to claim 17 wherein the retaining members are triangular in shape.
